(19) United States
(12) Patent Application Publication
(10) Pub. No.: US 2005/0112198 A1
(43) Pub. Date: May 26, 2005

(54) Bupropion formulation for sustained delivery

(76) Inventors: Prasad V.N. Challapalli, Lexington, KY (US); Peddanna Gumudavelli, Lexington, KY (US); Ram B. Murty, Lexington, KY (US)

Correspondence Address: Dr. Prasad V.N. Challapalli, Murty Pharmaceuticals, Inc., 518 Codell Drive, Lexington, KY, 40509, US

(21) Appl. No.: 10/975,618

(22) Filed: Oct. 27, 2004

Related U.S. Application Data
(60) Provisional application No. 60/514,722, filed on Oct. 27, 2003.

(51) Int. Cl. A61K 9/20

(52) U.S. Cl. 424/464

(57) ABSTRACT

Disclosed is a pharmaceutical formulation for the stabilization and sustained delivery of an active pharmaceutical ingredient, such as the antidepressant, bupropion.

### RELATED APPLICATIONS

Priority is claimed on the basis of U.S. nonprovisional application No. 60/514,722, filed Oct. 27, 2003.

### FIELD OF THE INVENTION

The invention relates to a pharmaceutical formulation for the stabilization and sustained delivery of an active pharmaceutical ingredient, such as the antidepressant, bupropion.

### BACKGROUND OF THE INVENTION

For many therapeutics which are administered orally, it is preferred that drug molecules be released into the body at a constant, or otherwise controlled rate, over a relatively long period of time, such as, for example, 4-8 hrs or longer. The primary objectives of a controlled release system have been to enhance safety and to provide an extended duration of action. Today, controlled release systems are designed in order to produce more reliable absorption and to improve bioavailability and efficiency of delivery.

Bupropion is an antidepressant agent that is chemically distinct from tricyclic and other commercially available antidepressants, e.g. selective serotonin reuptake inhibitors (SSRIs). Bupropion, described in U.S. Pat. Nos. 3,819,706 and 3,885,046, is currently available as the hydrochloride salt, i.e. as (±)-1-(3-chlorophenyl)-2-[(1,1-dimethylethyl) amino]-1-propanone hydrochloride, and is used as both an antidepressant (Wellbutrin®, Wellbutrin® SR and Wellbutrin® XL) and a smoking cessation aid (Zyban®). Utility in treating attention deficit hyperactivity disorder (ADHD) has also been evaluated. Bupropion hydrochloride is a water soluble, crystalline solid having a melting point of 233-234° C. It is highly hygroscopic and susceptible to decomposition (Walters, S. M., J. Pharm. Sci., 1980, 69(10), 1206-1209 and Laizure, S. C., et al Ther. Drug Monit., 1985, 7, 447-458). Because of the drug's instability, the shelf life of bupropion formulations has proven to be problematic, and those working in the field have tried a number of different approaches to improving the storage stability of the drug in formulations.

The typical daily dose of burpropion hydrochloride to treat depression is in the range of approximately 50 mg to 450 mg, preferably 200 mg to 300 mg. When used as an aid in smoking cessation (Zyban®), the typical daily dose is in the range of approximately 150 mg to 300 mg, available as extended release tablets. The preferred daily dosage regimen for treatment of depression is in the range of 50-300 mg, available either as an immediate release (Wellbutrin® 75 and 100 mg tablets) or as an extended release preparation meant for twice daily (Wellbutrin® SR, 50 mg-200 mg tablets) or once daily (Wellbutrin® XL, 150 mg and 300 mg tablets) administration. However, the exact dosage regimen will depend on a number of factors, including age, the general condition of the patient, and the particular condition or disorder.

The bupropion extended release preparations for twice daily or once daily administration may be considered to possess certain advantages over the immediate release dosage form in controlling seizures and in improved patient compliance. Bupropion may also preferable over other agents because of its minimal anticholinergic, cardiovascular and antihistaminic effects or in those patients who have experienced weight gain or sexual dysfunction with another antidepressant. Regardless of its available form, the stability of bupropion hydrochloride is affected by a number of factors, including formulation microenvironment and storage conditions. It is recommended that immediate and extended release tablets be stored in a tight container at 20-25° C. and also protected from light and moisture.

One of the requirements for an acceptable pharmaceutical composition is that it must be stable, so as not to exhibit substantial decomposition of the active ingredient during the time between manufacture and use by the patient. A number of drugs are known to undergo hydrolytic decomposition, which is one of the most common routes of drug decomposition. Further, hydrolytic decomposition can be influenced by light, oxidation and pH. For example, acid stabilization of the related compound diethylpropion hydrochloride has been described by Walters (1980) J. Pharm. Sci., 69 (10) 1206-1209. Placing bupropion hydrochloride in a relatively low pH environment has also proven to be effective in stabilizing bupropion and its major metabolites in human plasma; see Laisure et al. (1985) Ther. Drug Monit. 7(4): 447-450.

Certain of the art concerning bupropion formulations is discussed below.

In U.S. Pat. Nos. 5,358,970; 5,541,231; 5,731,000 and 5,763,493 to Ruff et al is described a stabilized bupropion hydrochloride formulation having a stabilizer selected from group consisting of L-cysteine hydrochloride, glycine hydrochloride, malic acid, sodium metabisulfite, citric acid, tartaric acid, L-cystine dihydrochloride, ascorbic acid, and isoascorbic(erythorbic) acid. U.S. Pat. No. 6,652,882 to Odidi et. al describes stabilization of drug by a saturated polyglycolised glyceride like Gelucire®.

The other acid stabilization strategies of bupropion formulation are achieved by inorganic acids like hydrochloric acid, phosphoric acid, nitric acid and sulfuric acid (U.S. Pat. No. 5,968,553); dicarboxylic acids like oxalic acid, succinic acid, adipic acid, fumaric acid, benzoic acid and phthalic acid (U.S. Pat. Nos.: 6,194,002; 6,221,917; 6,242,496; 6,482,987 and 6,652,882); sulfites like potassium metabisulfite and sodium bisulfite (U.S. Pat. No. 6,238,697); organic esters like L-ascorbic acid palmitate, tocopherol solution in alcohol, butylated hydroxy anisole, vitamin E succinate, vitamin E 700 acetate, and L-ascorbic acid G palmitate are used in transdermal preparations (U.S. Pat. No. 6,312,716). The use of acidified granules of microcrystalline cellulose (U.S. Pat. No. 6,153,223); salts of organic bases like creatinine hydrochloride, pyridoxine hydrochloride and thiamine hydrochloride and inorganic acid like potassium phosphate monobasic (U.S. Pat. No. 6,333,332) is also reported.

There are also acid-free stabilization strategies reported based on film coating, involving coating tablet cores with hydroxypropyl cellulose and ethyl cellulose (U.S. Pat. No. 6,306,436), or by a two layer coating step with ethyl cellulose, polyvinylpyrrolidone and eudragit L30D-55 (U.S. Pat. Nos. 6,033,686 and 6,143,327) or by giving multiple coatings onto drug-coated sugar pellets (U.S. Pat. No. 6,210,716), or a triple layer tablet prepared by melt granulation technique with carnauba wax, stearic acid and hypromellose mixture (U.S. patent application 2003/0134906) and by complexation with cyclodextrins (U.S. Pat. No. 6,462,237). A capsule dosage form having a mixture of three separately coated drug pellets to provide both immediate and extended release is also reported (U.S. Pat. No. 6,589,553).

Ludwig et al (U.S. Pat. No. 5,427,798) reported a hypermellose (HPMC)-based controlled released preparation of bupropion hydrochloride containing cysteine hydrochloride or glycine hydrochloride as stabilizer, and the release obtained is not less than 80% release in distilled water after 8 hrs.

Sustained release oral formulations of bupropion hydrochloride are prepared by mixing stabilized drug with various celluloses, alkyl and hydroxy celluloses, carboxy alkyl cellulose, polyalkylene glycols and acrylic acids (U.S. Pat. Nos. 6,110,973; 6,120,803; 6,153,223; 6,238,697; 6,333,332; 6,458,374 and 6,652,882) either individually or by combining with control release agents followed by coating the matrix (U.S. Pat. Nos. 6,210,716 and 6,462,237). It is also reported based on osmotic effects (U.S. Pat. Nos.: 4,769,027 and 4,687,660/Re 33,994) and sustained action through a transdermal patch (U.S. Pat. No. 6,312,716).

Ion exchange resin compositions containing pharmacologically active ingredients are known and such complexes often reported to have various pharmaceutical applications, primarily for taste masking, as release controlling agents in liquid forms, such as suspensions, and in tablet formulations, as disintegrants, and to a limited extent act as a controlled release agent.

An ion exchange resin is an ionic, or charged, compound which has binding sites that can bind or take up an ionic drug. The most common types of an ion exchange resins are polymers. The size of the ion-exchange resin should preferably fall within the range of about 20 to about 200 μm; particle sizes substantially below the lower limit are difficult to handle in all steps of the processing. Particle sizes substantially above the upper limit, e.g., commercially-available ion-exchange resins having a spherical shape and diameters up to about 1000 μm, are gritty in liquid dosage forms and have greater tendency to fracture when subjected to drying-hydrating cycles. Moreover, it is believed that the increased distance that a displacing ion must travel in its diffusion to displace drug out of these large particles causes a measurable but not readily controlled prolongation of release even when the drug/resin complexes are uncoated.

Amberlite® IRP-69, a cationic exchange resin (obtained from Rohm and Haas), is a sulfonated polymer composed of polystyrene cross-linked with 8% divinyl benzene, with an ion exchange capacity of about 4.5 to 5.5 meq/g of dry resin (H+ form). Amberlite® IRP-69 consists of irregularly shaped particles with a size range of 47 to 149 um, produced by milling the parent, large sized spheres of Amberlite® IRP-120.

In general, a drug is mixed with an aqueous suspension of the resin, and the complex is then washed and dried. Adsorption of drug onto the resin may be detected by measuring a change in the pH of the reaction medium or by other changes in physical properties or by a decrease in concentration of drug dissolved in the aqueous phase. Drug-resin complexes often dissolve more slowly than ordinary drug formulations. Such complexes are useful in changing dissolution profiles and are frequently used in time-release formulations. The release of drug can be extended further either by coating of a drug-resin complex or treating the complex with polymeric materials.

Adsorption of the drug onto the ion exchange resin particles to form the drug/resin complex is a well known technique as reported in U.S. Pat. Nos. 2,990,332 and 4,221,778. Akkaramongkolporn et. al (Drug Dev. Ind. Pharm., 2001, 27(4), 359-364) and Sriwongjanya et. al (Eur. J. Pharm. Biopharm. 1998, 46, 321-327, Chem. Pharm. Bull., 2000, 48(2), 231-234 ) described studies involving physical mixture of drug and resin in comparison to conventional drug-resin complex; Sriwongjanya et. al. also discussed presenting such complexes along with suitable excipients to achieve sustained action of drug from the compressed matrix.

Liquid pharmaceutical compositions comprising a drug bound to an ion exchange resin have been known for many years. Currently, the list of such commercially available extended release suspensions from Celltech Pharma, USA, includes Tussionex® (Hydrocodone polistirex/Chlorpheniramine polistirex), Delsym® (Dextromethorphan polistirex), and Codaprex® (Codeine polistirex and Chlorpheniramine polistirex). Other commercially available products include nicotine, as nicotine polacrilex in chewing gum (Nicorette®, GSK) and lozenges (Commit®, GSK), and phentermine in capsules (Ionamin® Celltech Pharma, USA). For instance, British patent 869,149 discloses the adsorption of ionizable drugs on ion exchange resins and the dispersion of the resultant solids in a carrier liquid, and German patent, 2,246,037 discloses drug adsorbed on resin particles, which loaded resin particles are then coated with a water-insoluble film-forming resin such as a polyacrylic acid ester. Sellassie et al in EP 249,949 have disclosed solid compositions comprising ion exchange resins.

Other publications disclose further treatment of drug-ion exchange resin particles 20 in order to improve sustained release characteristics. For example, U.S. Pat. No. 4,221,778 to Raghunathan disclosed ion exchange resin drug complexes treated with an impregnating agent such as polyethylene glycol or propylene glycol prior to coating with a water-permeable diffusion barrier such as ethylcellulose, the impregnating treatment is said to prevent swelling and fracturing of the resin particle in solutions and to improve coating ability. EP 171,528 and U.S. Pat. No. 4,847,077, to the same inventor, disclose a similar resin treatment using glycerine to improve coating ability. EP 254,811 and EP 254,822, both to Raghunathan and Chow, disclose similar impregnation of sulfonic cation exchange resin with agents such as hypromellose (HPMC) and with high molecular weight polymers, to improve coating ability. U.S. Pat. Nos. 5,071,646 and 6,077,532 reported the use of a sugar solution as granulating fluid to improve the strength of the drug-resin particles. EP 139,881 and U.S. Pat. No. 4,762,709 by Shumaker discloses compositions in which all ionic components having the same charge are present as resin complexes, thereby overcoming variations in the dissolution profile caused by ionic substances in the formulation.

A dextromethorphan hydrobromide controlled release syrup suspension was achieved by complexing with Amberlite® IRP-69 (U.S. Pat. No. 4,788,055), and a similar preparation was also reported involving coating drug-complex particles followed by mixing with uncoated drug/resin material (U.S. Pat. No. 6,001,392). Various liquid preparations based on ion exchange resins have also been reported (U.S. Pat. Nos. 4,762,709; 5,368,852 and 6,514,492).

A stable sustained release wax and polymer-coated drug-ion exchange resin complex useful in preparing oral suspension has also been reported (U.S. Pat. Nos. 4,999,189 and 5,186,930). Drug-resin complexes coated with water insoluble polymers to achieve sustained action for oral administration are reported (U.S. Pat. Nos. 4,996,047; 5,288,503; 5,413,782 and U.S. patent application 2003/0099711). A pharmaceutically acceptable preservative and a chelating agent have also been added to effect drug-resin complex stabilization (U.S. Pat. Nos.: 4,894,239; 5,368,852; 5,980,882 and 6,001,392). A sustained release opioid preparation without any coating has also been reported (U.S. patent applications 2002/0164373 and 2003/0118641). Various ophthalmic preparations based on ion exchange resin compositions have also been reported (U.S. Pat. Nos. 5,182,102; 5,296,228; 5,540,918; 5,837,226; 6,022,533 and 6,258,350).

In-house studies showed that the salt form of bupropion is stable in normal storage conditions but can degrade easily in the presence of standard excipients used in commercial formulations. The presence of such excipients can contribute significantly to increase the overall moisture level and hence facilitate forced degradation of the active pharmaceutical agent unless it is protected. An object of the invention is therefore to increase the stability of active drug while controlling its release from the dosage form to obtain a desired release profile.

### BRIEF DESCRIPTION OF THE DRAWINGS

FIG. 1 shows a comparative dissolution test profiles of twice-daily tablet formulations according to the invention (Bupropion HCl SR and Bupropion XR (resinate), 150 mg each) vs. Zyban® (150 mg) in water.

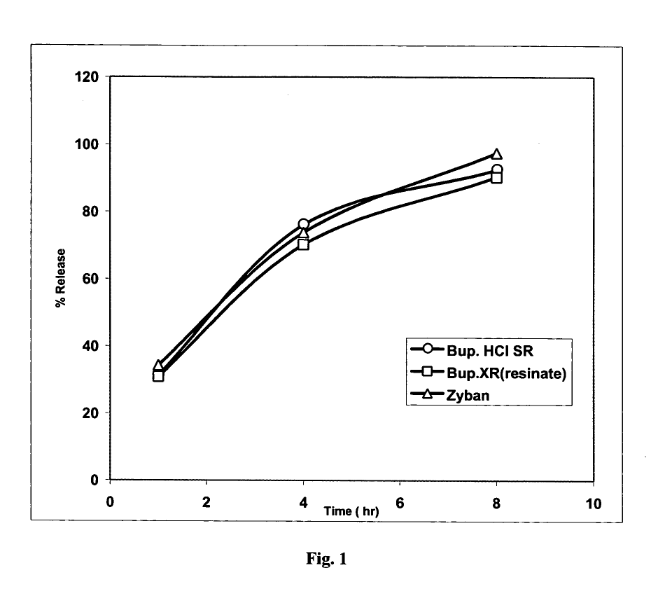

FIG. 2 shows a comparative dissolution test profile of a once-daily tablet formulation according to the invention (Bupropion HCl XR, 150 mg) vs. Wellbutrin® XL (150 mg) in mixed acid and phosphate buffer medium.

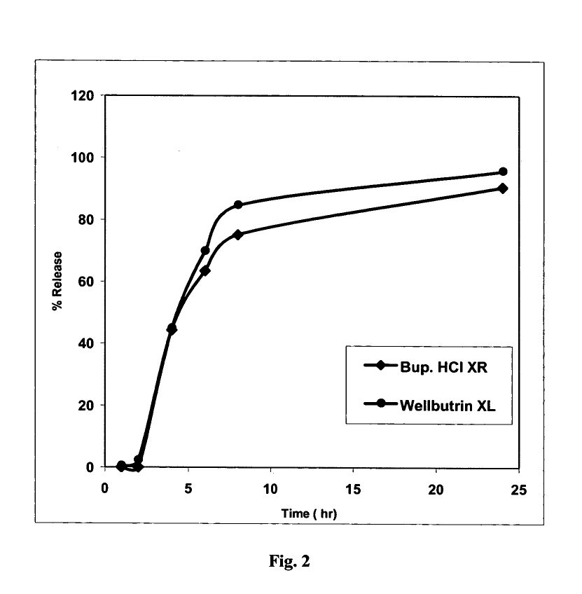

### SUMMARY OF THE INVENTION

An object of the invention is to provide a bupropion hydrochloride pharmaceutical composition preferably for controlled or sustained release that is stabilized alone or in combination of an added stabilizer(s) or by an cation exchange resin. Protection may be conferred by an additional coat surrounding the stabilized/un-stabilized core wherein the film optionally contains stabilizers to facilitate both enhanced stability to the preparation and to provide control over the release of the active ingredient, where it contains at least about 80% potency of undegraded burpropion hydrochloride after storage for three months at about 40° C. and 75% RH.

Stabilization of the active ingredient, according to the invention, comprises, for example, any of the following four processes:
(i) physical mixing or treating burpropion hydrochloride with a pharmaceutically acceptable stabilizer(s) either alone or in combination of suitable stabilizers in an effective amount. The method utilizes either dry blending or granulation of bupropion hydrochloride with suitable pharmaceutical excipients, stabilizer and agent(s) for controlled release of the drug.
(ii) the addition of an ion exchange resin particles to the aqueous solution of bupropion hydrochloride to facilitate the formation of drug-resin complex and dried. The complex achieved is mixed with suitable pharmaceutical excipients and a controlled release agent(s) either by physical mixing or by granulation followed compression/filling to achieve a stable and sustained release dosage form.
(iii) physical mixing or treatment of ion exchange resin particles with other suitable pharmaceutical excipients and chosen stabilizer(s) alone or in combination, optionally included the active drug also for such dry mixing or granulation and dried. The treated mixture has been compressed/filled in to tablets/capsules by mixing with suitable glidants and lubricants to achieve desired release.
(iv) coating of the stabilized or un-stabilized dosage form with a film surrounding the core which optionally may have added stabilizer(s) to offer additional control over its release and in maintaining the stability of the preparation during its storage.

An object of the invention is to provide a release of bupropion hydrochloride from uncoated/film coated bupropion HCl SR dosage form, of not less than 25% in 1 hr, not more than 85% in 4 hr and not less than 80% in 8 hr in a dissolution test conducted at 37° C. in distilled water as per USP 27. Bupropion HCl SR and bupropion HCl XR dosage forms when tested in 0.1N HCl buffer, the release after 1 hr should be not more than 50%, after 4 hr should not be more than 85%, and after 8 hr should not be less than 80%. Another object of the invention includes providing a release of bupropion hydrochloride from enteric coated bupropion HCl XR dosage form, less than 10% in 2 hr, not more than 60% in 4 hr, not less than 60% in 8 hr and not less than 80% in 16 hr in a dissolution test conducted at 37° C. similar to the procedure mentioned for enteric coated tablets as per USP 27.

An object of the invention is to provide a release of bupropion from uncoated/film coated bupropion XR resinate formulation, of not more than 50% after 1 hr, not more than 85% after 4 hr, after 8 hr should not be less than 80% in a dissolution test conducted at 37° C. similar to the procedure mentioned for enteric coated tablets as per USP 27. From the bupropion XR resinate dosage form when enteric coated the release of bupropion should be less than 10% in 2 hr, not more than 60% in 4 hrs, not less than 60% in 8 hrs and not less than 80% in 16 hr in a dissolution test conducted similar to the procedure mentioned for enteric coated tablets as per USP 27.

The invention is described below in connection with its applicability to bupropion. However, the invention relates additionally to the formulation of other pharmaceuticals, such as any acidic or basic drug, which can be bound to an ion exchange resin or can be stabilized by chosen stabilizer(s) alone or in combination for the purpose of stabilizing the formulation or to mask the taste or any other useful applications known in that art with such combination(s). The invention is particularly well suited to water-soluble drugs, but is useful for sparingly water-soluble drugs.

A formulation according to the invention is also suitable to achieve desired drug release pattern for any drug which is soluble or slightly soluble by carefully manipulating the chosen controlling agents along with other suitable excipients where such type of release is recommended for its intended pharmacological action or to limit any drug related side effects or any known benefits it offers to the patient. The dosage form is also include a suitable coating film surrounding the core to offer additional assurance either in terms of release or making the preparation more stable during its storage.

### DETAILED DESCRIPTION OF THE INVENTION

The invention provides, for example, a stable formulation of bupropion hydrochloride that releases the medicament in a controlled manner after oral administration to provide a desired therapeutic effect to a human patient. Such a formulation comprises, for example, a core consisting of an active drug, a stabilizer, a controlled release agent, a pharmaceutically acceptable diluent, a glidant, and a lubricant.

Unless otherwise stated, the following terms have the meaning set forth below. Notwithstanding the foregoing, should any of the meanings set forth below conflict with the meaning of that term as that term is conventionally used in the pharmaceutical arts, the meaning of that term as that term is conventionally used in the pharmaceutical arts shall control.

The term “optional” or “optionally” means that it may or may not be obvious about its addition to the formulation however it can be added as the situation demands depending on the release pattern aimed from the dosage form.

The term “pharmaceutically acceptable” excipients means a material may be administered to an individual along with the active agent without causing any undesirable biological effects or interacting in a deleterious manner with any of the other components of the pharmaceutical composition in which it is contained and these includes, diluents, binders, lubricants, fillers, coloring agents, stabilizers, ion exchange resins, plasticizers, opacifiers and other such agents known in the art.

By the term “therapeutically effective amount” of an agent as provided herein are meant a non-toxic but sufficient amount of the agent to provide the desired therapeutic effect. The exact amount required will vary from subject to subject depending on age, general condition of the subject, the severity of the condition being treated.

The term “controlled release” is intended to refer to any drug-containing formulation in which release of the drug is not immediate i.e., with “a controlled release formulation”, oral administration does not result in immediate release of the drug into absorption pool.

The term “sustained release” is used in its conventional sense to refer to a drug formulation that provides for gradual release of drug over an extended period of time, which results in substantially constant blood levels of drug over an extended period of time.

“Enteric coating” or “enterically coated” as used herein relates to the presence of polymeric materials in a drug formulation that results in an increase in the drug resistance to disintegration in the stomach.

The term “resinate” which refers to any drug-resin complex that would have formed by a process in which drug gets loaded onto any resin preferably in presence of a solution. Such complex is washed at the end of loading, dried and stored as needed.

According to the invention, an active pharmaceutical agent, such as bupropion hydrochloride, is presented in a dosage form in any of the following manners: (a) drug is in association of stabilizer(s) alone or in combination of suitable stabilizers and/or with excipients treated or granulated with stabilizers; (b) drug is in association of ion exchange resin which is complexed to form a resinate mixture and further granulated with suitable binder(s) either alone and/or with suitable excipients; (c) drug is co-mixed with ion exchange resin and suitable excipients followed by treating or granulating such mixture with a solution of stabilizer(s); (d) drug is directly mixed or granulated with suitable excipients.

In-house studies showed that the salt form of bupropion is stable in normal storage conditions but can degrade easily in the presence of standard excipients used in commercial formulations. The presence of such excipients can contribute significantly to increase the overall moisture level and hence facilitate forced degradation of the active pharmaceutical agent unless it is protected. An object of the invention is therefore to increase the stability of active drug while controlling its release from the dosage form to obtain a desired release profile.

Oxidation of organic materials has been widely known in the area of drug research and is usually studied with help of pure systems or simple mixtures at extreme conditions. It is usually difficult to predict causes of such behavior in a complex pharmaceutical system with vastly different physicochemical properties where drug can exist in crystalline or amorphous form along with excipients of different physical nature. This mixture is often subject to various mixing methods like trituration, blending, compaction or compression process to induce more physical changes into the overall matrix properties. Oxidation in pure solid requires that an oxidizing agent be present in the system for reaction to occur. Molecular oxygen from the atmosphere has been shown to react with organic materials. This reactivity depends on the morphology of crystal form, which governs the permeability and solubility of oxygen in the crystal matrix. In some cases, reactivity decreases with increasing melting point suggesting that stronger crystal lattice energies inhibit oxygen diffusion. Free radicals are known to form in crystalline solids, which can react with molecular oxygen to form reactive and diffusible oxygen species such as superoxide anion or peroxyl radical. These reactive species can abstract hydrogen atoms at reactive positions on drugs or induce electron transfer from a drug (Waterman K. C., et. al. Pharm. Dev. Technol., 2002, 1-32).

In many cases like the bupropion hydrochloride, pure drug substance is quite stable, while the dosage form shows significant drug oxidation. This difference in reactivity is generally attributable to amorphous nature of drug or moisture content of the excipient or due to the impurities present in the excipient. Firstly, during the processing of drug to form a solid dosage form, it is possible to mechanically generate a small percentage of amorphous drug to cause stability issues. Amorphous drug regions have both greater mobility and high energy but lack crystal lattice stabilization energy, and as a result, oxygen permeability and solubility will be higher. Any fortuitous radical-pair initiation is more likely to lead free radicals, which can react with oxygen in a chain process to make the degradation process faster. Secondly, excipeints can potentially solvate some of the drug either directly or by bringing in low levels of moisture. In a solid solution form, the drug will be amorphous and such solvation step can result in drug deprotonation to make a drug more reactive and unstable. Thirdly, the excipients can be a source of oxidants and metals act often as catalysts rather than consumed even at low levels. Typically, iron and copper are the two metals of most concern. Alternatively, oxidation is also related to highly reactive impurities like peroxides, superoxides, hypochlorites and formic acid present in the excipients as manufacturing related impurities (Hartauer, K. J., et. al. Pharm. Dev. Technol., 2000, 5, 303-310). Mostly, the peroxides and other oxidants are introduced during the polymer manufacturing process including the polymer initiation and purification. Peroxides are used sometimes as initiators in polymerization and are difficult to remove completely during the purification processes. For example, it is easily found in polyethylene glycols, tweens and polyvinylpyrrolidone. In some cellulose based excipeints, a peroxide or hypochlorite bleaching process is used that can allow trace peroxides or hypochlorite to remain in the excipient. In the case of polyethylene oxides, high molecular weight material is prepared and then oxidatively degraded to give the desired molecular weight range. This degradation leads to peroxides and other low molecular weight species such as formate ions. Aldehyde impurities can also induce drug oxidation in some cases. In the solid state, such chain propagation is hindered by low mobility and such reactions tend to occur over extremely short distances with fewer chain propagation steps.

The possible strategy to terminate such propagations includes the addition of chelating agents to the formulation to prevent metal induced oxidation. These include citric acid, edetic acid, fumaric acid and malic acid. They can be more effective when added during granulation step than in a physical mix form. In cases, where peroxides are believed to be the problem it can be overcome by enforcing strict quality measures at the time of choosing them or to restrict usage of such excipients to minimum possible amounts in the dosage form. Use of antioxidants, which can reduce formation of peroxides will be helpful but may be less effective at elimination of peroxides already present in a dosage form. Currently, the marketed bupropion hydrochloride(Zyban® and Wellbutrin® SR) is stabilized with an antioxidant like L-cysteine hydrochloride. The chain lengths as mentioned are generally shorter in solid dosage forms than in liquids, the concentration of antioxidant may need to be higher to intercept radicals. Sacrificial reductants like sulfites and ascorbic acid are compounds that are oxidized more readily to scavenge the oxygen than the drug. This strategy can be especially effective in systems where the amount of oxygen present is limited due to packaging. In some cases, this class of antioxidants can be problematic in the presence of metals since the drug reducing agents can sometimes reduce metals to more reactive forms. Therefore, enough precautions should be taken to handle such situations.

The environment of pH can play a significant role in the stabilization of drugs to oxidation. It is in general more difficult to remove an electron from a drug when it is positively charged. For this reason, drug stability against oxidation is often greater under low pH conditions, which promote protonation of drugs if porotonation is possible. In the converse, higher pH conditions, which deprotonate a drug, generally make the drug more susceptible to oxidation. With the solid dosage forms, the addition of buffers can be quite complex. In order them to be more effective it is advisable to add them during wet granulation or by coating of particles using fluid bed technology.

The success of any sustained release or controlled delivery of a drug is achieved by using polymeric excipients to control the rate that an active drug is introduced to the targeted delivery site. The exact mechanism by which a polymer controls the delivery of the drug is dependent on the rate of polymer hydration and swelling which is related to its molecular weight. Therefore, any process that significantly reduces the molecular weight of the polymer is likely to affect its ability to control the drug delivery. Oxidative degradation can lead to a loss in molecular weight for several polymers commonly used in controlled release applications. In addition to loss in molecular weight such degradation in polymers as mentioned can produce reactive impurities and end groups to compromise the chemical stability of drugs and also their effectiveness as release controlling agents. An example of class of controlled release polymers that can degrade to compromise the drug release rate is the polyoxyethylenes, including poly(ethylene oxides) (Polyox®), poly (ethylene glycols), and poly (oxyethylene) alkyl ethers. The polyethylene oxide has been treated by manufacturer (Dow chemicals) with 100-1000 ppm of butylated hydroxy toluene (BHT) to reduce such degradation. This antioxidant is quite effective, however, it is volatile and can be lost during any heating steps. In the proposed invention, an additional amount of butylated hydroxyl toluene is used to minimize such loss and is also combined with butylated hydroxy anisole (BHA) or propyl gallate to obtain synergistic action.

Appropriate packaging can represent a reasonable option for stabilization of the drug against molecular oxygen and light. When drug instability especially caused by atmospheric oxygen, the amount of headspace available and oxygen permeation through the container walls and caps of the packaging system plays a vital role. For most plastic containers, the rate of oxygen permeation can be significant. For example, low density polyethylene (LDPE) and polystyrene container are more permeable than high density polyethylene (HDPE), polypropylene, polyvinyl chloride, polycarbonate, polyethylene terephthalate (PET), wherein the rate of permeation decreases in the order they were listed. The oxygen leakage through container top can be countered by heat induction sealing. The above approaches are appropriate for maintaining product stability until the containers are opened for the first time for dispensing, and it is possible for a sensitive product to lose its characteristics thereafter. To avoid such situations, especially in sensitive products like orally or rapidly disintegrating tablets, a blister packaging of individual doses is preferred, even though the process is not cost effective but it can offer better product stability. It is sometimes feasible to blister package under nitrogen or argon to reduce the head space oxygen.

In order to study the compatibility of various excipients used in the proposed invention with bupropion hydrochloride, various drug-excipient mixtures were prepared. The each excipient amount relative to drug varied from 1:1 to 1:10. Such mixtures were prepared, by mixing them in a pestle and mortar, and transferred to glass vials and either closed with a rubber stopper or studied in an open condition at 50° C./75% RH and 40° C./75% RH. As predicted with the active drug, it has been observed during their analysis at various time intervals by liquid chromatography that most of the excipients studied are causing degradation of the active drug with a result of two distinct degradation peaks, in which all cellulose based excipients considered to be interacting uniquely with the pure drug compared to non-cellulose based excipients. In the case of hydroxypropyl cellulose, polyethylene oxide and sodium carboxymethyl cellulose, rapid degradation was found relative to other excipients, with noticeable visual/odor/color changes in the prepared mixtures. The above changes were more pronounced in samples studied under open conditions compared to closed condition samples. A minimum amount or no change was observed with polyvinyl acetate-polyvinylpyrrolidone coplymers (Kollidon® VA64 and Kollidon® SR), methylcellulose, colloidal silicon dioxide, magnesium stearate, enteric coating materials such as hypromellose pthalate, hypromellose succinate and polyvinylacetate phthalate. Such degradation could be due to impurities like peroxide, heavy metals and bleaching agents present in such materials. Even though such impurities are present within acceptable limits, the moisture content carried in them should have acted as solvent to aggravate such reactions. In addition, the proportions according to the invention are slightly higher than the required amounts in the actual dosage form but certainly the above information helps in minimizing their usage quantities to make the dosage form more stable. Hence, pure drug is treated with chosen stabilizers and continued the remaining studies under closed conditions. The package component seems to play a vital role for stabilizing the dosage form: high-density polyethylene (HDPE) containers with additional thickness/weight are preferred apart from maintaining sufficient quantity of adsorbent added to the packaging system to minimize moisture and oxygen/odor levels.

Protection of a drug molecule according to the invention includes not only minimization of potentially deleterious drug-excipient interactions, by making drug molecule more stable by treating with various stabilizers, but also presentation of drug molecule in a suitable complex or physically mixed form with ion exchange resin, in addition to packaging of the final product in a packaging system with desiccants having sufficient adsorbent capacity to offer resistance to permeation of moisture, oxygen, light and by any other agents known to degrade the molecule.

An ion-exchange resin is a useful excipient according to the invention, as bupropion hydrochloride is capable of forming a complex with a cation exchange resin for both stabilization and sustained release. Protection of the drug with various stabilizers involves a mechanism of shielding the drug molecule from degradation through the use of reductants, chelators, or substances helpful in maintaining a low pH environment recommended for drug stability by various reported studies (Walters, S. M., J. Pharm. Sci., 1980, 69(10), 1206-1209).

Ion-exchange resins suitable for use in these preparations are water-insoluble and consist of a pharmacologically inert organic or inorganic matrix containing covalently bound functional groups that are ionic or capable of being ionized under appropriate conditions of pH.

A wide range of cationic (for basic drugs) or anionic (for acidic drugs) exchange resin can be used to form a drug-resin complex, with particle sizes normally ranging from about 75 to 1000 um. Illustrative examples employ Amberlite IRP69, which is a milled product of 100-200 mesh size resin particles of Amberlite IR-120, as a model small particle resin. It is a gel type divinylbenzene sulfonic acid cation exchange resin, which swells in water with a pH range of 0-14. Ion-exchange resins useful according to the invention have exchange capacities below about 6 meq/g and preferably below about 5.5 meq/g.

Drug-resin complex was formed by a step-wise method, where resin particles, of proportion 0.25 to 2.0 parts relative to 1 part of drug, were added at each step to an aqueous drug solution having an initial concentration of about 100-300 mg/ml, and the contents stirred for 2-4 hr until equilibrium was attained in the solution. Samples collected at various intervals were allowed to settle, filtered, and diluted with water for measurement of absorbance by UV spectrometry at 298.0 nm. Drug-resin mix solution was allowed to settle at the end of each step and decanted into a separate container. A quantity of fresh resin, in appropriate proportion, was added to the decanted solution to adsorb the remaining amount of drug; these steps were repeated until complete adsorption of drug from the solution onto the resin had taken place. Usually, complete drug adsorption (>98%) onto resin was achieved in 3-4 steps. To achieve that, the total added resin quantity varied anywhere from 0.5 to 5.0 parts relative to 1 part of drug. The drug loaded resin particles collected at various stages were combined and washed in distilled water by stirring for 2-4 hr to remove loosely/surface bound drug molecules, and such amounts were estimated by measuring absorbance of the wash solution. The washed particles were dried overnight at 50° C. and stored in a tight container; assay was performed by HPLC to confirm the percentage of drug loading onto resin. It has been observed from applicants' experience that stepwise loading of drug onto resin particles gave better adsorption yields than adding all the required amount of resin at once to the drug solution. The dried resinate is useful for making controlled release formulations according to the invention. The ratio of drug to resin in such a complex is from about 1:0.5 to 1:5, and preferably from about 1:0.5 to about 1:3.

In certain formulations, ion exchange resin is also added directly to the free form of a drug, without any complex formation, to achieve formulations performing similarly to the resinate under certain circumstances, thus greatly simplifying the manufacturing and regulatory aspects, in comparison to resinate-based formulations. Formulations made by such direct addition may release drug more rapidly, due to weak drug-resin bonding, but release characteristics are also manipulated with various controlled release agents according to the invention to achieve the desired release from the dosage form. The ratio of drug to resin in such a physical mix is from about 1:0.01 to about 1:0.5, and preferably from about 1:0.03 to about 1:0.4.

Among the ion exchange resins useful in the present invention are styrene-divinyl benzene copolymers (e.g. IRP69, IRP120, IRP400 and IRP67, Rohm & Haas), copolymers of methacrylic acid and divinylbenzene (e.g. IRP 64 and IRP88, Rohm & Haas), phenolic polyamines (e.g. IRP58, Rohm & Haas) and styrene-divnylbenzene (e.g., colestyramine resin) and similarly various type of resins available as DOWEX® series (Dow Company). The drug and resin should be oppositely charged such that the drug will bind to the resin in the dissolved drug solution. Since bupropion hydrochloride is a basic drug, it is preferred that the ion exchange resin for a buproprion formulation according to the invention be cationic in nature, and more preferably be strongly acidic in nature. For formulation of another active pharmaceutical ingredient, is possible to use other types of ion exchange resins such as anionic type (e.g. duolite AP 143, Rohm & Haas) known in the art depending on the specific needs of application and drug nature. A preferred ion exchange resin for a buproprion formulation according to the invention is Amberlite IRP 69 or any other equivalent form of resin.

The invention provides a stabilized bupropion formulation for oral administration; said formulation comprises stabilizers to inhibit or prevents the degradation of active drug. Stabilizers useful in accordance with the present invention maintain at least about 80% of the potency of the drug and preferably over 90% of potency after three months storage at 40° C./75% RH. The mechanism of stabilizing the active drug by chosen stabilizers other than drug-resin complex form involves shielding the drug molecule by their anti-oxidative property, chelating action or they are helpful in maintaining a low pH environment around the drug molecule. The chosen stabilizers were preferably solubilized in a suitable solvent system such as water, hydro-alcoholic mixtures or in non aqueous solvents and used to treat the drug particles or excipients. In some situations, such as when the stabilizer is hydrophobic in nature, treatment/coating of drug particles with stabilizer solutions is found to influence the solubility/release of the drug in dissolution medium. In those conditions, a solution of such stabilizer is used initially to treat/coat the suitable excipients of the formulation and further granulated/blended with other ingredients and active drug as needed. Stabilizers are often directly mixed with active drug and other excipients of the formulation to achieve a controlled release dosage form. Preferred stabilizers according to the invention are butylated hydroxy anisole (BHA), butylated hydroxy toluene (BHT), toluene sulfonic acid, disodium edetate, edetic acid, lactic acid, maleic acid, propyl gallate, sodium formaldehyde sulfoxylate, benzoic acid and aspartic acid. It is possible to use other stabilizers known in the art depending on the specific needs of the particular application and the nature of the drug. The proportion of stabilizer(s) in a formulation according to the invention is from about 0.001% to about 5% w/w, and preferably from about 0.001% to about 3% w/w.

A stabilized active drug molecule, formed by mixing active drug either with ion-exchange resin or with other stabilizers in the form of treated/coated, complexed or physically mixed form, according to a procedure as described above, is used for making a controlled release formulation of the active drug molecule after mixing with pharmaceutically acceptable excipients. The mass of active pharmaceutical agent, whether it be in a pure form, a salt form or in a complex, ranges from 25 mg to 300 mg. The proportion of active pharmaceutical agent in a final dosage form, said final dosage form consisting essentially of active pharmaceutical agent, excpients, and any adventitious materials, is from about 10% to about 80% w/w, and preferably from about 15% to about 60%.

A resinate-based formulation of bupropion hydrochloride according to the invention is preferably granulated with a binder to improve the hardness of the formulation. Resinate particles are very fine and hence not easily compressible, especially when the resinate proportion is more than 60% w/w in the formulation; some segregation of materials may occur, due to different particle sizes of other excipients added to the final blend. In some cases, it is possible to achieve some minimum hardness without granulation by addition of binders of a certain type, which was found to contribute significantly to overall hardness of the final product. But this makes it difficult to achieve the final target weight of the product, as they have to be added in sufficiently large quantities to achieve such desired objective. Otherwise, they will fail to withstand a coating process for lack of sufficient hardness.

The invention also provides a formulation of bupropion hydrochloride comprising an ion exchange resin, the formulation manufactured by admixing free buproprion with the ion exchange resin and a suitable binder, controlled release agent, glidant and lubricant. Optionally, such a formulation is granulated and a pore forming agent is also included, depending on the release requirements of the designed dosage form.

When the active drug is stabilized with other than ion exchange resins, the stabilizier-treated active drug is mixed with a diluent/binder, a controlled agent and either blended or granulated with an aqueous, hydro-alcoholic or non-aqueous solvent mixture as demanded by the properties of the chosen materials. Optionally, an ion exchange resin or a pore former is also included.

A binding agent according to the invention is water soluble, and should possess high adhesivity and an appropriate viscosity, to guarantee good adhesion between the drug/resinate particles and other added excipients of the formulation. According to the invention, such a binding agent also functions as a diluent in that it acts to impart cohesive qualities to the material within the formulation and also to increase the bulk weight of the directly compressible formulation, to achieve an acceptable formulation weight for direct compression. In situations where there is a need for immediate availability of active drug, a pore-forming agent is also included in the dosage form. A main function of such a pore-forming agent is to create a pore in the matrix predominantly due to the freely soluble nature of such an agent in aqueous medium or in the gastrointestinal contents.

Therefore, a diluent according to the invention possesses the property of being a binder or a pore-forming agent. The following materials commonly known in the art to perform/exhibit such function include starch, pregelatinized starch, microcrystalline cellulose, silicified microcrystalline cellulose, dibasic calcium phosphate anhydrous, powdered cellulose, alginic acid, povidone, chitosan, carageenan, guar gum, xanthan gum, polyethylene oxide, substituted copolymes of polyvinylacetate-polyvinyl pyrrolidone, stearic acid, carnauba wax, polacrilin resin, lactose, mannitol, maltitol, maltose, fructose, xylitol, trehalose, sorbitol, dextran, dextrins, dextrose, maltodextrin, polyethylene glycol, carboxymethylcellulose sodium, carboxymethylcellulose calcium, polyvinyl alcohol, hydroxypropyl cellulose, hydroxypropyl ethylcellulose, hydroxyethyl cellulose, hydroxyethyl methylcellulose, methylcellulose and ethylcellulose. The total percentage of their addition to the formulation as binder either individually or in combination is from about 1% to about 70% w/w, and preferably from about 5% to about 55% w/w. The total percentage of optional pore former either individually or in combination is from about 1% to about 30% w/w, and preferably from about 2% to about 10% w/w.

The extended or controlled release of active drug from a formulation according to the invention is achieved using individual or a combination of excipients known in the art to perform as barrier-forming polymer(s), erodable or insoluble material(s). Such excipients include hydroxy propyl cellulose, hydroxypropyl ethyl cellulose, hydroxyethyl cellulose, hydroxyethyl methy cellulose, sodium carboxymethylcellulose and calcium carboxymethylcellulose, polyethylene oxides of molecular weight 100,000-7,000,000 Daltons(Polyox®), polyvinyl alcohols (MW: 20,000-200,000 Daltons), substituted copolymers of polyvinyl acetate and polyvinylpyrrolidone like kollidon® VA64 and kollidon® SR, sodium alginate, carrageenan, xanthan gum individually or in combination of ceratonia, locust bean gum or veegum, guar gum, gellan gum, methylcellulose and chitosan, ethylcellulose, dextrates, dextrins, eudragit® (RL and RS grade), cellulose acetate, cellulose acetate trimellitate, cellulose acetate butyrate, cellulose acetate propionate, cetostearyl alcohol, cetyl alcohol, glyceryl behenate derivatives like compritol® 888 ATO, precirol® ATO 5, gelucire® 44/14, gelucire® 50/13, glyceryl mono oleate, glyceryl mono stearates, glyceryl palmito stearates, lecithin, medium chain triglycerides, eudragit® RSPO, eudragit® RLPO, stearic acid, stearyl alcohol, hydrogenated vegatable oil, carnauba wax, microcrystalline wax and beeswax. The total amount of controlled release agent present in the dosage form either alone or in combination is from about 5% to about 75% w/w, and preferably from about 5% to about 60% w/w.

Drug release is controlled by variables such as surface area and diffusion rate in a dynamic process. Barrier-forming polymers form a dynamic hydrophilic matrix system to allow for slow release of drug in a patient's body. Upon exposure to water, these barrier-forming high viscosity materials will hydrate and swell rapidly to form a hydrogel, which expands with time into the interior of the tablet allowing for diffusion of the drug from the tablet core slowly to facilitate for controlled drug delivery. Further, such effects are supported by insoluble materials of type swellable or non-swellable in nature, which can acts as barrier or shielding the drug molecule under their hydrophobic coat to discourage the drug diffusion out of gelled matrix and to an extent it also helps in slowing down the matrix erosion process and thus delaying the overall dissolution rate of drug from the dosage form

A preferred glidant or lubricant according to the invention is colloidal silicon dioxide, talc, magnesium stearate, calcium stearate, stearic acid or sodium stearyl fumarate. The proportion of glidant and lubricant present in the dosage form is from about 0.5% to about 3% w/w.

The manufacturing process for the core is either by wet granulation, dry granulation or direct compression, and where applicable involves mixing the necessary ingredients of the respective formulations according to the strategies discussed earlier, preferably in a high shear mixer granulator (GMI, India) or planetary mixer (Hobert, USA) to obtain homogeneity. If necessary, the drug is mixed with diluents prior to any granulation step. In such case, the premixed blend is then granulated with water or any suitable granulation fluid and dried in a fluidized bed dryer (Gansons, India) or tray dryer (Kothari, India) as required. Alternatively, heat can be applied during granulation to the level where binder, filler melts and components mixed well to get particles coated. The dried granular mass is milled and then mixed with any other excipient (such as any diluent or controlled release agent) not added during granulation. Glidants are blended with the resulting mixture in a V-blender (Patterson-Kelley, USA) or double cone blender (Gansons, India) or octagonal blender (Gansons, India) which is capable of functioning under preferred low shear conditions followed by addition of lubricants. The lubricated mass is then compressed into tablets using a tablet press (Cadmach, India) or filled into capsule shells of suitable size using a semi-automatic capsule filling machine (Pam, India) or manual filling machine (Pam, India). The capsule shell preferably comprises gelatin, starch, chitosan or hypromellose.

A dosage form core according to the invention is preferably film-coated with a coating material such as hydroxypropyl cellulose, a methacrylate copolymer such as eudragit® E100 or eudragit® EPO, kollicoat® IR, opadry® II or opadry® AMB for identification, taste masking, aesthetic purpose and for stability. According to the invention, the film coating acts as a moisture barrier. The film coating does not substantially affect the release rate of drug from the tablet/capsule, since the coating is of instant release, which rapidly dissolves in the stomach. Many polymers have been investigated for use in film-coating. Most film coats are prepared by deposition of one or more film-forming polymers resulting in coats that usually represent no more than about 2-5% by weight of the final coated product. A coating solution according to the invention preferably contains, in addition to the film-former, a plasticizer, a glidant and an opacifying agent or a coloring agent, and a solvent system therefor is composed of aqueous, hydro-alcoholic or non aqueous solvent mixture with stabilizer(s), as described above, optionally added to such a coating solution. Some coating materials are readily available in the form of a premix with all the necessary agents required for achieving a smooth and uniform film with necessary amount of deposition to protect the core against the moisture. The preferred amount of each coating material is as follows: film coating material, from about 5% to about 20% w/w; plasticizer, from about 0.1% to about 2% w/w; opacifying agent, from about 0.1% to about 10% w/w; glidant/antitacking agent, from about 0.1% to about 10% w/w; optional stabilizer, from about 0.001% to about 3% w/w.

A dosage form core according to the invention is preferably enteric coated to retard release during the initial period of transit through the gastrointestinal system. Such enteric coat is applied either directly onto the core of the dosage form or applied over a film coat, which can not only act as sub-coat to protect the dosage form against moisture but also helps in binding of the enteric film firmly and uniformly to the dosage form. Examples of suitable enteric polymers include hypromellose phthalate, hypromellose acetate succinate, cellulose acetate phthalate, cellulose acetate trimelliate, polyvinyl acetate phthalate based dispersions like Opadry® enteric and Sureteric® either directly or after mixing with suitable plasticizers, glidants, opacifying agents or coloring agents wherein the coating solutions is prepared in aqueous, hydro-alcoholic or non aqueous solvent mixtures with stabilizer(s) optionally added to such coating solutions.

In coating solutions other than readily available, incorporation of suitable plasticizers into the polymer matrix effectively reduces the glass transition temperature, so that under ambient conditions the films are softer, more pliable, and often stronger, and thus better able to resist the mechanical stress, which otherwise forms a hard, non-pliable and rather brittle, properties which could be somewhat restrictive in film coating since the coated dosage form may be subjected to a certain amount of external stress. Examples of suitable plasticizers include dibutyl sebacate, diethyl phthalate, triethyl citrate, tributyl citrate, castor oil and triacetin.

A preferred plasticizer, opacifying agent and glidant according to the invention are triethyl citrate, titanium dioxide and talc respectively, which are added to a coating solution prepared freshly by combining them with necessary film forming materials apart from suitable coloring agents. The preferred amount of each coating material is as follows: enteric coating material, from about 5% to about 20% w/w; plasticizer, from about 0.1% to about 2% w/w; opacifying agent, from about 0.1% to about 10% w/w; glidant/antitacking agent, from about 0.1% to about 10% w/w; optional stabilizer, from about 0.001% to about 3% w/w.

A dosage form core according to the invention is coated in a pan coater (Ganscoater, India). An aqueous or a mixture of an organic and aqueous solvent or a mixture of organic solvent is used for film or enteric coating. Examples of suitable organic solvents are e.g., ethanol, methanol, methylene chloride, isopropyl alcohol and with or without water. The dosage form core is coated until appropriate weight gain achieved like 1-4% w/w in the case of film coating and approximately 5-15% w/w for enteric coated tablets. The tablets are dried or allowed to curing as needed at the end of each coating process. The operational parameters are maintained according to the manufacturer recommendations.

A buproprion hydrochloride formulation according to the invention, for sustained or controlled release delivery of bupropion hydrochloride, is suitable for either twice daily or once daily administration as needed by the therapy or the patient need. A dosage form according to the invention is, for example, a tablet or a capsule containing from about 25 mg to about 300 mg of active drug and is evaluated for performance in dissolution and assay at various intervals during stability studies. Stability studies are conducted as per the ICH guidelines. The dissolution test is conducted using USP Type II (Hansons Research, USA) apparatus. The twice daily formulations of bupropion are tested using water as dissolution medium at 37° C. Once-daily bupropion uncoated tablets/capsules are tested at 37° C. in 0.1 N HCl, and their coated dosage forms are tested at 37° C. according to the procedure recommended by USP27 for enteric-coated dosage forms. Bupropion resinate based formulations either for once daily or twice daily administration are tested according to the procedures described for enteric coated tablets in USP 27. The above procedure with resinate based formulations is necessary to simulate the gastro-intestinal conditions as the release from such dosage form is dependent directly on the number of ions available either in dissolution medium or in gastrointestinal fluids.

Any of a wide variety of therapeutically active agents is formulated into a formulation according to the present invention. A therapeutically active agent according to the invention is a water soluble drugs or a slightly water soluble drug. A therapeutically active agent according to the invention is, for example, any hydrochloride salt form of amosulalol, aclarubicin, amantadine, amiloride, betazolol, benazepril, bisoprolol, buspirone, barnidipine, bifemelane, bepridil, clofedanol, cetrizine, cevimeline, chloperastine, cephalexin, cyclobezaprine, diltiazem, dobutamine, dibucaine, dexamethylphenidate, ethambutol, etafenone, esmolol, fluoxetine, flurazepam, fexofenadine, granisetron, hydralazine, indenolol, loperamide, levamisole, lomefloxacin, metformin, methylphenidate, metoclopramide, moperone, metixene, mefloquine, nortriptyline, nicardipine, ondansetron, oxycodone, oxymorphone, paroxetine, promethazine, propafenone, quinapril, ritoridine, ranitidine, rimantadine, sertraline, ticlopidine, tolazoline terazosin, tamsulosin, tramadol, tiapride, tolazoline, terbinafine, tirofiban, verapamil, valacyclovir, or venlafaxine or any salt form of abacavir sulfate, alendronate sodium, citalopram hydrobromide, clopidogrel bisulfite, cerivastatin sodium, chloroquine phosphate, diclofenac sodium, diclofenac potassium, enalapril maleate, enoxaprin sodium, fluvastatin sodium, fosinopril sodium, indinavir sulfate, losartan potassium, naproxen sodium, neostigmine bromide, oxybutynin chloride pravastatin sodium, primaquine phosphate, piperacillin sodium, quetiapine fumarate, risedronate sodium, rosiglitazone maleate, valporate sodium, salbutamol sulfate, sumatriptan succinate, warfarin sodium or zolpidem tartrate.

The following materials have been used in preparing embodiments of the invention but can be replaced with similar materials available commercially and known in the art.
Bupropion Hydrochloride (Dipharma, Italy)
Amberlite® IRP 69 (Rohm & Hass)
Hydroxypropyl cellulose (HPC, Klucel®, EXF and HXF grade, Hercules)
Polyethylene oxide (Polyox® 303 WSR grade, Dow Chemicals)
Hydroxyethyl cellulose (HEC, Natrosol®, 250 HHX grade, Hercules)
Ethylcellulose (ECT 10 grade, Hercules)
Sodium carboxymethy cellulose (7HXF grade, Hercules)
Polyvinylacetate-polyvinylpyrrolidone copolymer (Kollidon® SR and Kollidon® VA64 from BASF)
Cellulose acetate butyrate (CAB, Eastman)
Compritol® ATO 888 and Glucire® 50/13 (Gattefosse)
Polyvinyl alcohol (Sigma)
Lactose anhydrous, DC grade (DCL 21, JRS Pharma)
Microcrsyalline cellulose (Avicel PH102, FMC)
Mannitol (Mannogem, SPI Pharma)
Colloidal Silicon Dioxide (Cab-o-sil, Cabot)
Magnesium stearate (Malinkrocdt)
Opadry® II and Opadry® AMB(both from Colorcon)
Opadry® enteric and Sureteric® (both from Colorcon)
Hypromellose phthalate and hypromellose succinate (Shinetsu)
Eudragit® RSPO, Eudragit® RLPO, Eudragit® E100, Eudragit® EPO (all form Degussa)
Kollicoat® IR (BASF)
Butylated hydroxy anisole (BHA) and butylated hydroxy toluene (BHT) (Both from Sigma)

### EXAMPLES

The following are examples, specifically with respect to a sustained release dosage form of bupropion hydrochloride. The formulation details of bupropion hydrochloride 150 mg tablets are disclosed. Capsule formulations are also readily prepared through the use of similar excipient amounts. A tablet or capsule formulation containing between about 25 mg and about 300 mg bupropion or bupropion hydrochloride is manufactured by a method similar to that disclosed in the examples and in a dose-proportional manner or by using excipients and procedures as disclosed in the examples and adapted as per the release requirements. Such a formulation evinces a dissolution profile and a release profile substantially similar to that disclosed herein.

Bupropion HCl SR 150 mg Tablets (Twice Daily)

### Example-1

| | Qty (mg/tab) | |
|---|---|---|
| Bupropion HCl | 150 | |
| Microcrystalline cellulose | 210 | |
| Xanthan Gum | 40 | |
| Cab-o-sil | 1 | |
| Magnesium stearate | 3 | |
| Butylated hydroxy anisole | 5 | |
| Butylated hydroxy toluene | 0.4 | |
| Tablet weight | 409.4 | mg |
| Pore former (optional) | 25 | |
| Tablet weight | 434.4 | mg |

Procedure: Stabilizers were dissolved in small quantities of ethyl alcohol and were used to treat the blend of microcrystalline cellulose and xanthan gum by mixing them for 10 min. The granules were dried at 50° C. Dried and milled granules were mixed for about 10 min with sifted bupropion hydrochloride, cab-o-sil and optional pore former. The above granules were lubricated for 2 min and compressed into tablets using tablet press.

Optionally, the treatment of granules is performed with hydro-alcoholic (1:1) mixture or includes the bupropion hydrochloride part of core granulation.

A formulation similar to the above is manufactured without BHA or BHT in the core but with a film coating as described in example 11 or example 12.

### Example-2

| | Qty (mg/tab) | |
|---|---|---|
| Bupropion HCl | 150 | |
| Hydroxyethyl cellulose (250 HHX) | 80 | |
| CR agent # 1 | 55 | |
| Cab-o-sil | 1 | |
| Magnesium stearate | 3 | |
| Butylated hydroxy anisole | 5 | |
| Butylated hydroxy toluene | 0.4 | |
| Tablet weight | 294.4 | mg |

Procedure: Stabilizers were dissolved in small quantities of ethyl alcohol and were used to treat the blend of hydroxyethyl cellulose by mixing them for 10 min and dried at 50° C. The dried granules were milled and blended for 10 min with bupropion hydrochloride and the CR agent(s) #1.The agent(s) selected were added either individually or in combination of one or more chosen from the following like carnauba wax, eudragit® RSPO, compritol® ATO 888, gelucire® 50/13, cellulose acetate butyrate and polyvinyl alcohol. Finally, the mixed granules were blended for 10 min with sifted cab-o-sil followed by lubrication for 2 min and compressed into tablets using tablet press. The treatment of granules can also be done with hydro-alcoholic (1:1) or non-aqueous solvent mixture and dried. Optionally CR agent #1 and bupropion hydrochloride can be included in the core granulation.

A formulation similar to the above is manufactured without BHA or BHT in the core but with a film coating as described in example 11 or example 12.

### Example-3

| | Qty (mg/tab) | |
|---|---|---|
| Bupropion HCl | 150 | |
| Ion exchange resin | 25 | |
| Polyox (WSR 303) | 80 | |
| CR agent #2 | 35 | |
| Cab-o-sil | 3 | |
| Magnesium stearate | 3 | |
| Butylated hydroxy anisole | 5 | |
| Butylated hydroxy toluene | 0.4 | |
| Tablet weight | 301.4 | mg |

Procedure: The ion exchange resin used was Amberlite® IRP-69. Stabilizers were dissolved in small quantities of ethyl alcohol and treated the blend of resin for 5 min and dried at 50° C. The dried and milled granules were mixed for 10 min with bupropion hydrochloride, polyox and CR agent #2. The CR agent(s) selected were added either individually or in combination of one or more chosen from the following like kollidon® SR, kollidon® VA 64 and ethylcellulose (ECT10). Finally, the mixed granules were blended for 10 min with sifted cab-o-sil followed by lubrication for 2 min and compressed into tablets using tablet press. Alternatively, CR agent #2 and bupropion hydrochloride can be included in the core granulation. The above granulation can also be carried out with purified water and dried at 50° C.

A formulation similar to the above is manufactured without BHA or BHT in the core but with a film coating as described in example 11 or example 12.

Bupropion XR(Resinate) 150 mg Tablets (Once/Twice Daily)

### Example-4

| | Qty (mg/tab) | |
|---|---|---|
| Bupropion- resinate | | |
| eq. to active bupropion HCl | 150 | |
| CR agent #3 | 15 | |
| HPC (EXF) | 100 | |
| Cab-o-sil | 6 | |
| Magnesium stearate | 6 | |
| Tablet weight | 577 | mg |

Procedure: Bupropion-resinate mixture, binder and controlled release agent were sifted and mixed well for 10 min. The CR agent(s) selected were added either individually or in combination of one or more chosen from the following like HPC (HXF), eudragit® RSPO, polyox® (WSR 303), sodium carboxymethyl cellulose (7 HXF) and hydroxyethyl cellulose (250 HHX). Further, mixing was done after the addition of sifted cab-o-sil for 10 min. The above granules were lubricated for 2 min and compressed into tablets using a tablet press. Alternatively, the above granulation was carried out with purified water and granules were dried at 50° C.

### Example-5

| | Qty (mg/tab) | |
|---|---|---|
| Bupropion- resinate | | |
| eq. to active bupropion HCl | 150 | |
| Polyox ® (WSR 303) | 60 | |
| Kollidon ® SR | 100 | |
| Cab-o-sil | 5 | |
| Magnesium stearate | 5 | |
| Tablet weight | 620 | mg |

Procedure: Bupropion-resinate mixture, polyox® and kollidon® SR were sifted, and mixed well for 10 min. Sifted cab-o-sil was added to above granules and mixed for 10 min. The above granules were lubricated for 2 min and compressed into tablets using a tablet press.

### Example-6

| | Qty (mg/tab) | |
|---|---|---|
| Bupropion- resinate | | |
| eq. to active bupropion HCl | 150 | |
| Polyox ® (WSR 303) | 50 | |
| Kollidon ® VA64 | 50 | |
| CR agent # 4 | 50 | |
| Cab-o-sil | 6 | |
| Magnesium stearate | 6 | |
| Tablet weight | 612 | mg |

Procedure: Bupropion-resinate mixture, kollidon® VA 64 was granulated with purified water by mixing them for 10 min and dried at 50° C. The dried and milled granules were mixed with sifted polyox and additional controlled release agent and mixed well for 10 min. The CR agent(s) #4 selected were added either individually or in combination of kollidon® SR and ethylcellulose (ECT10). Further, mixing was done after the addition of sifted cab-o-sil for 10 min. The above granules were lubricated for 2 min and compressed into tablets using a tablet press.

Bupropion XR(Resinate) 150 mg Capsule (Once/Twice Daily)

### Example-7

| | Qty (mg/cap) | |
|---|---|---|
| Bupropion- resinate | | |
| eq. to active bupropion HCl | 150 | |
| Xanthan gum | 25 | |
| HEC(250 HHX) | 25 | |
| Cab-o-sil | 6 | |
| Magnesium stearate | 6 | |
| Tablet weight | 512 | mg |

Procedure: Bupropion-resinate mixture, xanthan gum and HEC were sifted and mixed for 10 min. Further, mixing was done after the addition of sifted cab-o-sil for 10 min. The above granules were lubricated for 2 min and filled into a capsule of suitable size using manual capsule filling machine.

Bupropion HCl XR 150 mg Tablets (Once Daily)

### Example-8

| | Qty (mg/tab) | |
|---|---|---|
| Bupropion HCl | 150 | |
| Ion exchange resin | 37.5 | |
| HPC (EXF) | 62.5 | |
| CR agent #5 | 62.5 | |
| Cab-o-sil | 3 | |
| Magnesium stearate | 3 | |
| Butylated hydroxy anisole | 5 | |
| Butylated hydroxy toluene | 0.4 | |
| Tablet weight | 323.9 | mg |

Procedure: The ion-exchange resin used was Amberlite® IRP-69. Stabilizers were dissolved in small quantities of ethyl alcohol and were used to treat the mixed blend of resin and HPC (EXF) for 5 min and dried at 50° C. Dried and milled granules were mixed with burpropion hydrochloride and CR agent #5 for 10 min. The CR agent(s) #5 selected were added either individually or in combination of one or more chosen from the following like HPC (HXF), polyox® (WSR 303), HEC (250 HHX). The above granules were mixed with sifted cab-o-sil for 10 min followed by lubrication for 2 min and compressed into tablets using tablet press. Alternatively, they were also granulated with inclusion of bupropion hydrochloride and CR agent #5 with ethyl alcohol or mixture of water:ethanol (1:1) solution and dried.

A formulation similar to the above is manufactured without BHA or BHT in the core but with a film coating as described in example 11 or example 12.

### Example-9

| | Qty (mg/tab) | |
|---|---|---|
| Bupropion HCl | 150 | |
| Ion exchange resin | 25 | |
| CR agent # 6 | 40 | |
| Polyox (303 WSR) | 100 | |
| Cab-o-sil | 3 | |
| Magnesium stearate | 3 | |
| Butylated hydroxy anisole | 5 | |
| Butylated hydroxy toluene | 0.4 | |
| Tablet weight | 326.4 | mg |

Procedure: The ion-exchange resin used was Amberlite® IRP-69. Stabilizers were dissolved in small quantities of ethyl alcohol and were used to treat the blend of resin for 5 min and dried at 50° C. The dried granules were milled and mixed with bupropion hydrochloride, polyox and CR agent #6 for 10 min. The CR agent(s) selected were added either individually or combination of one or more chosen from the following like ethylcellulose (ECT10), kollidon® SR and kollidon® VA64. The blend was mixed for 10 min with sifted cab-o-sil followed by lubrication for 2 min and compressed into tablets using tablet press. Alternatively, the compositions not in combination with kollidon® SR can be granulated with ethyl alcohol and a mixture of water:ethanol (1:1) and dried. A formulation similar to the above is manufactured without BHA or BHT in the core but with a film coating as described in example 11 or example 12.

### Example-10

| | Qty (mg/tab) | |
|---|---|---|
| Bupropion HCl | 150 | |
| Ion exchange resin | 25 | |
| Kollidon ® SR | 120 | |
| Polyox (303 WSR) | 50 | |
| Kollidon ® VA 64 | 50 | |
| Cab-o-sil | 4 | |
| Magnesium stearate | 4 | |
| Butylated hydroxy anisole | 5 | |
| Butylated hydroxy toluene | 0.4 | |
| Tablet weight | 408.4 | mg |

Procedure: The ion-exchange resin used was Amberlite® IRP-69. Stabilizers were dissolved in small quantities of ethyl alcohol and were used to treat the mixed blend of resin and kollidon® VA 64 for 5 min. Alternatively, they were also granulated with mixture of water: ethanol (1:1) or ethanol solution and dried at 50° C. Dried and milled granules were mixed with bupropion hydrochloride, polyox, kollidon® SR and cab-o-sil for 10 min. The above granules were lubricated for 2 min and compressed into tablets using tablet press.

A formulation similar to the above is manufactured without BHA or BHT in the core but with a film coating as described in example 11 or example 12.

Film Coating

The examples described in examples #1-9 were film coated using in-house prepared coating solution or using opadry® II, opadry® AMB, Eudragit® EPO or kollicoat® IR coating dispersion system. The details of the in-house system were mentioned below

### Example-11

| | Qty (mg/tab) |
|---|---|
| Core tablets/caps | |
| (examples #1-10) | |
| Film Coating material | 10% w/w |
| Plasticizers | 0.5% w/w |
| Opacifying agent | 0.5% w/w |
| Glidants/antitacking agent | 1.0% w/w |
| Solvent(s) mixture | |
| Optional | 1.0% w/w |
| Stabilizers | |
| Total weight gain | 4.0% w/w |

Procedure: The aqueous/non-aqueous/hydroalcoholic coating solution was prepared and coated by using convenient film coating dispersion systems like kollicoat® IR or opadry® II/opadry® AMB or Eudragit® EPO as per the instructions provided by manufacturer to achieve the required target weight. The in-house aqueous/non-aqueous coating solution was prepared by using suitable film coating materials like hydroxypropyl cellulose and Eudragit® E100 along with necessary film forming agents mentioned in example 11. The core units were coated in a Ganscoater 250 (Bombay, India) with necessary temperature settings ideal for respective coating materials and were dried for 30-60 min after achieving the required weight gain. Alternatively, the stabilizers are dissolved in a suitable solvent and mixed with coating solution to coat the tablets/capsules.

Enteric Coating

The examples described from #4-10 were optionally enteric coated over a film coated dosage form described in example 11, using materials like hypromellose phthalate or hypromellose acetate succinate or using polyvinyl acetate phthalate (opadry® enteric or sureteric® coating dispersion system). The coating solutions were prepared either in aqueous or in non-aqueous solvent system. The readily dispersible coating solutions were prepared as per the instructions provided by manufacturer. The details of the in-house system were mentioned below

### Example-12

| | Qty (mg/tab) |
|---|---|
| Core tablets/caps | |
| (examples #4-10) | |
| Enteric coating material | 10% w/w |
| Plasticizers | 0.7% w/w |
| Opacifying agent | 0.3% w/w |
| Glidants/anti-tacking agent | 0.6% w/w |
| Solvent(s) mixture | |
| Optional | 1.0% w/w |
| Stabilizer | |
| Total weight gain | 8.0% w/w |

Procedure: The aqueous/non-aqueous/hydroalcoholic enteric coating solution was prepared and coated by using convenient coating dispersion systems like Sureteric® or Opadry® enteric as per the instructions provided by manufacturer to achieve the required target weight. The in-house aqueous/non-aqueous coating solution was prepared by using suitable enteric coating materials like hypromellose phthalate or hypromellose acetate succinate along with necessary film forming agents mentioned in example 12. The core units were coated in a Ganscoater 250 (Bombay, India) with necessary temperature setting ideal for respective coating materials and were dried for 30-60 min after achieving the required weight gain. Alternatively, the stabilizers are dissolved in a suitable solvent and mixed with coating solution to coat the tablets/capsules.

Dissolution Studies

Dissolution studies were performed at 37° C.

Bupropion HCl 150 mg SR Tablets (Twice Daily)

The prepared bupropion hydrochloride 150 mg SR tablets (examples: 1-3) are tested for dissolution in a USP Type II apparatus at 50 rpm in 900 ml of pure water and the percentage release data is shown in Table 1.

TABLE 1
Dissolution data for bupropion HCl 150 mg SR Tablets

| Time (hr) | Example #1 | Example #2 | Example #3 |
|---|---|---|---|
| 1 | 37.2% | 31.2% | 31.2% |
| 4 | 72.8% | 66.0% | 76.1% |
| 8 | 89.7% | 89.1% | 92.5% |

Bupropion XR(Resinate) 150 mg Tablets (Twice Daily)

The prepared bupropion XR(resinate) 150 mg tablets (examples: 4-6) are tested for dissolution in USP Type II apparatus at 75 rpm in 750 ml of 0.1N HCl for 2 hours of 250 ml of 0.2M phosphate buffer (pH 6.8) and the percentage for twice daily tablets is shown in Table 2.

TABLE 2
Dissolution data for bupropion XR (resinate)
150 mg Tablets (Twice daily)

| Time (hr) | Example #4 | Example #5 | Example #6 |
|---|---|---|---|
| 1 | 39.6% | 30.9% | 25.5% |
| 4 | 75.3% | 70.2% | 73.2% |
| 8 | 86.4% | 90.3% | 94.3% |

Bupropion XR(Resinate) 150 mg Capsules (Once Daily)

The prepared bupropion XR(resinate) 150 mg capsules (example :7) is tested for dissolution in USP Type II apparatus at 75 rpm in 750 ml of 0.1N HCl for 2 hours followed by addition of 250 ml of 0.2M phosphate buffer (pH 6.8) and the percentage release data obtained for once daily uncoated capsule is shown in Table 3.

TABLE 3
Dissolution data for bupropion XR (resinate) 150 mg capsules

| Time (hr) | Example #7 |
|---|---|
| 2 | 35.4 |
| 4 | 58.4 |
| 6 | 63.7 |
| 18 | 85.1 |
| 24 | 89.3 |

Bupropion HCl XR150 mg Tablets (Once Daily)

The prepared bupropion XR 150 mg tablets (examples: 8-10) are tested for dissolution in USP Type II apparatus at 75 rpm either in 750 ml of 0.1N HCl for 2 hours followed by addition of 250 ml of 0.2M phosphate buffer (pH 6.8) or tested in 900 ml of 0.1N HCl and the percentage release data is shown in Table 4 and 5.

TABLE 4
Dissolution data for bupropion HCl XR 150 mg
Tablets in 0.1 N HCl and Phosphate buffer

| Time (hr) | Example #8 | Example #9 | Example #10 |
|---|---|---|---|
| 1 | 0.0 | 0.0 | 0.0 |
| 2 | 0.1 | 2.0 | 0.0 |
| 4 | 33.3 | 54.7 | 44.4 |
| 6 | 51.3 | 88.8 | 63.5 |
| 8 | 64.6 | 95.5 | 75.2 |
| 24 | 86.6 | — | 90.6 |

TABLE 5
Dissolution data for bupropion HCl
XR 150 mg Tablets in 0.1 N HCl

| Time (hr) | Example #8 | Example #9 | Example #10 |
|---|---|---|---|
| 1 | 24.3 | 30.4 | 29.3 |
| 2 | 39.1 | 47.5 | 43.0 |
| 4 | 67.1 | 73.1 | 61.6 |
| 6 | 80.3 | 87.4 | 75.0 |
| 8 | 89.6 | 92.8 | 82.4 |

Stability Studies

The stability studies are conducted for above illustrated examples (1-10) of bupropion hydrochloride dosage form meant for once/twice daily administration by storing them at 40° C./75% RH for 3 months. The samples are analyzed by an high performance liquid chromatography system (HP 1100, Agilent, USA) with a gradient method using C₈ column (Phenomenex, 4.6×100 column, 3.5 um) having flow rate of 1.5 ml/min at wavelength of 226 nm and mobile phase consists of combination of solvent mixture A (water:acetonitrile:trifluroacetic acid, 900:100:0.4 ml) and mixture B (acetonitrile:water and trifluorocacetic acid, 950:50:0.3 ml). The assay results have shown that all the formulation have maintained above 90% potency of the active drug during storage. The drug-resinate blend stored at 40° C./75% RH for 6 months maintained above 90% of the active drug.

Further Embodiments

The invention provides additionally the following further embodiments:

1. A pharmaceutical solid tablet dosage form consisting of bupropion hydrochloride and any acceptable pharmaceutical salts of bupropion base in which the drug and/or its formulation is suitably stabilized either alone or in combination of an added stabilizer(s) and/or by an ion exchange resin with suitable excipients in a physically mixed, treated or complexed form and/or by a suitably coated film surrounding the core which optionally have added stabilizer in the film and such preparation intended to release the drug in a desired manner to provide an controlled delivery of the drug as needed by the patient or the therapy.

2. The solid dosage form mentioned in Further Embodiment 1,
a. can be a tablet or a capsule dosage form.
b. the pharmaceutically acceptable excipients selected from the group consisting of diluents, release controlling agents, glidants and lubricants and mixtures thereof.
c. the diluent of 2(b) is also a binder and a pore former.
d. the core is surrounded by coating layer with stabilizers added optionally to its film.

3. The dosage form mentioned in Further Embodiment 2 wherein the active material
a. is in association of stabilizer(s) alone or in combination of stabilizers and/or with excipients suitably treated or granulated with stabilizers.
b. is in association of ion exchange resin which is complexed to form a drug-resinate mixture and further granulated with suitable binder(s) either alone and/or with suitable excipients.
c. is co-mixed with ion exchange resin and suitable excipients followed by treating or granulating such mixture with a solution of stabilizer(s).
d. is directly mixed or granulated with suitable excipients.

4. The dosage form mentioned in Further Embodiment 3 wherein the active materials is treated or granulated directly with aqueous/non aqueous solvents or hydro-alcoholic mixture thereof.

5. The dosage form mentioned in Further Embodiment 3 wherein the stabilizer(s) dissolved in aqueous/hydro-alcoholic or non-aqueous solvent(s) and such solution is used to treat granules directly or mixed/diluted with aqueous/hydro-alcoholic mixture solutions thereof and granulated.

6. The solid dosage form mentioned in Further Embodiment 2, wherein the dosage form is manufactured by any of the methods like direct compression, direct filling, dry granulation or by wet granulation.

7. The solid dosage form mentioned in Further Embodiment 6, wherein the dosage form is manufactured by any method like direct compression or dry granulation using binder(s) to prevent capping of the formulation.

8. The solid dosage form mentioned in Further Embodiment 6, is manufactured by wet granulation using binder(s) to offer both enough strength to granules and also to make non-compressible drug-resin complex particles to compress them easily.

9. The dosage form mentioned in Further Embodiment 6, wherein the binder(s) used can be aqueous and non-aqueous soluble binder(s).

10. The solid dosage form mentioned in Further Embodiment 6 wherein binder(s) used can be either polymeric or non-polymeric nature and mixture thereof.

11. The solid dosage form mentioned in Further Embodiment 2 consisting of bupropion hydrochloride or any pharmaceutically acceptable salt thereof, stabilizer(s) alone or in combination, controlled release agent(s), diluents, glidants and lubricants.

12. The solid dosage form mentioned in Further Embodiment 2 consisting of bupropion hydrochloride or any pharmaceutically acceptable salt thereof along with ion exchange resin existing in a complex form along with necessary controlled release agent(s), diluents, glidants and lubricants.

13. The solid dosage form mentioned in Further Embodiment 2 consisting of bupropion hydrochloride or any pharmaceutically acceptable salt thereof, stabilizer(s) or ion exchange resin alone or in combination with suitable controlled release agent(s), diluents, glidants and lubricants.

13B. The solid dosage form mentioned in Further Embodiment 2 consists of bupropion hydrochloride or any pharmaceutically acceptable salt thereof with suitable controlled release agent(s), diluents, glidants and lubricants.

14. The solid dosage form mentioned in Further Embodiment 2, the drug stabilization is carried out either in a mixed or treated form using stabilizer(s) such as butylated hydroxy anisole, butylated hydroxy toluene, toluene sulfonic acid, disodium edetate, edetic acid, lactic acid, maleic acid, propyl gallate, sodium formaldehyde sulfoxylate, benzoic acid and aspartic acid, and above were used either individually or in combination to achieve drug stability.

15. The solid dosage form mentioned in Further Embodiment 14, wherein the total stabilizer(s) percentage amount in formulation ranging from 0.001% to 5% w/w, more precisely the range is in between 0.001% to 3% w/w.

16. The solid dosage form mentioned in Further Embodiment 14, the drug stabilization is carried out by combination of butylated hydroxy anisole and butylated hydroxy toluene wherein any of them is mixed with the other stabilizer in a ratio of 1:0.01 to 1:20, especially the ration is between 1:1 to 1:15.

17. The solid dosage form mentioned in Further Embodiment 2, the drug is stabilized with an ion exchange resin capable of exchanging either anionic or cationic group.

18. The solid dosage form mentioned in Further Embodiment 17, the drug is stabilized with a resin capable of exchanging cationic group like amberlite® IRP 69 or Dowex® series.

19. The solid dosage from mentioned in Further Embodiment 17, the ratio proportion between drug and resin complex is ranging from 1:0.5 to 1:5, more appropriately it ranges from 1:0.5 to 1:3.

20. The solid dosage from mentioned in Further Embodiment 17, the ratio between the drug and resin in a physical mix is ranging from 1:0.01 to 1:0.5, more preferably from 1:0.03 to 1:0.4.

21. The diluent(s) mentioned in Further Embodiment 2 are used alone or in combination from the following microcrystalline cellulose, silicified microcrystalline cellulose, dibasic calcium phosphate anhydrous, powdered cellulose, alginic acid, povidone, chitosan, carageenan, guar gum, xanthan gum, polyethylene oxide, substituted copolymes of polyvinylacetate-polyvinylpyrrolidone, stearic acid, carnauba wax, polacrilin resin, lactose, mannitol, maltitol, xylitol, trehalose, sorbitol, polyethylene glycol, carboxymethylcellulose sodium, polyvinyl alcohol, carboxymethylcellulose calcium, hydroxypropyl cellulose, hydroxypropyl ethyl cellulose, hydroxyethyl cellulose hydroxyethylmethyl cellulose, methylcellulose and ethylcellulose.

22. The diluent(s) mentioned in Further Embodiment 2, the following perform as binder(s) in any combination of microcrsyatlline cellulose, dibasic calcium phosphate anhydrous, xanthan gum, carnauba wax, povidone, polyethylene oxide, substituted copolymers of polyvinylacetate-polyvinylpyrrolidone, hydroxypropyl cellulose, hydroxypropyl ethyl cellulose and hydroxyethyl cellulose, carboxymethylcellulose sodium, methylcellulose and ethylcellulose.

23. The diluents(s) mentioned in Further Embodiment 2, the following added optionally as pore former to the dosage form in any combination of lactose, mannitol, polyethylene glycol, polyethylene oxide, microcrystalline cellulose, polacrilin potassium and hydroxyethyl cellulose.

24. The diluent(s) mentioned in Further Embodiment 2, the total percentage of their addition to the formulation as binder either individually or in combination is ranging from 1% to 70%, more specifically in the range of 5% to 55% w/w.

25. The solid dosage form mentioned in Further Embodiment 2, the diluent(s) like hydroxypropyl cellulose, hydroxyethyl cellulose, polyvinylacetate-polyvinylpyrrolidone copolymer and polyethylene oxide alone or in combination added to the formulation maintained in total amounts of either below 20% w/w or their total amounts maintained above 40% w/w.

26. The diluent(s) mentioned in Further Embodiment 2, the total percentage of optional pore former either individually or in combination present in the range of 1 to 30% w/w, more preferably from 2% to 10% w/w.

27. The solid dosage form mentioned in Further Embodiment 2, the release controlling agent is added either alone or in combination of any of barrier forming polymer(s), erodable or insoluble material(s).

28. The controlled releasing agent in Further Embodiment 27, the total amount of such agent either alone or in combination ranging from 5% to 75% w/w, more specifically from 5% to 60% w/w.

29. The controlled releasing agent in Further Embodiment 27, consists either individually or in combination of barrier forming polymers of alkyl hydroxy cellulose like hydroxy propyl cellulose, hydroxypropyl ethyl cellulose, hydroxyethyl cellulose, hydroxyethylmethy cellulose, salts of polycarboxyalkyl cellulose like sodium carboxymethylcellulose and calcium carboxymethylcellulose, polyethylene oxide of molecular weight 100,000-7,000,000 Daltons(Polyox®), polyvinyl alcohols (MW: 20,000-200,000 Daltons), substituted copolymers of polyvinyl acetate and polyvinylpyrrolidone like kollidon® VA64 and kollidon® SR, sodium alginate, carrageenan, xanthan gum individually or in combination of ceratonia, locust bean gum or veegum, guar gum, gellan gum, methyl cellulose and chitosan.

30. The controlled release agent of Further Embodiment 27, chosen from a group comprising either individually or in combination of barrier forming polymers like hydroxy propyl cellulose, hydroxypropyl ethyl cellulose, hydroxyethyl cellulose, hydroxyethylmethy cellulose, sodium carboxymethylcellulose, polyethylene oxide of molecular weight 100,000-7,000,000 Daltons(Polyox®), substituted copolymers of polyvinyl acetate and polyvinylpyrrolidone like kollidon® VA64 and kollidon® SR, xanthan gum individually or in combination of gellan gum, locust bean gum or veegum, guar gum and methylcellulose.

31. The controlled release agent of Further Embodiment 27, consists either individually or in combination of erodible materials of alkyl hydroxyl celluloses like hydroxypropylcellulose, hydroxypropyl ethyl cellulose, hydroxyethyl cellulose, methylcellulose, ethylcellulose and hydroxyethyl methyl cellulose; salts of polycarboxyalkyl cellulose like sodium carboxymethylcellulose and calcium carboxymethylcellulose, polyethylene oxide of molecular weight 100,000-7,000,000 Daltons,(Polyox®), high molecular weight polyvinyl alcohols (MW: 20,000-200,000 Daltons), substituted copolymers of polyvinyl acetate and polyvinylpyrrolidone like kollidon® VA64 and kollidon® SR grade materials, polyethylene glycols (MW: 1000-35,000), dextrates, dextrins and eudragit® (RL and RS grade).

32. The controlled release agent of Further Embodiment 27, chosen from a group consists either individually or in combination of erodible materials like hydroxypropylcellulose, hydroxypropyl ethyl cellulose, hydroxyethyl cellulose, methylcellulose, ethylcellulose, sodium carboxymethylcellulose polyethylene oxide of molecular weight 100,000-7,000,000 Daltons, (Polyox®), high molecular weight polyvinyl alcohols (MW: 20,000-200,000 Daltons), substituted copolymers of polyvinyl acetate and polyvinylpyrrolidones are kollidon® VA64 and kollidon® SR grade materials and polyethylene glycols (MW: 1000-35,000).

33. The controlled release agent of Further Embodiment 27, consists either individually or combination of the insoluble materials like cellulose acetate, cellulose acetate trimellitate, cellulose acetate butyrate, cellulose acetate propionate, ethylcellulose, cetostearyl alcohol, cetyl alcohol, glyceryl behenate derivatives like compritol® 888 ATO, precirol® ATO 5, gelucire® 44/14, gelucire® 50/13, glyceryl mono oleate, glyceryl mono stearates, glyceryl palmito stearates, lecithin, medium chain triglycerides, eudragit® RSPO, eudragit® RLPO, stearic acid, stearyl alcohol, hydrogenated vegatable oil, carnauba wax, microcrystalline wax and beeswax.

34. The controlled release agent of Further Embodiment 27, chosen from a group consist either individually or in combination of insoluble materials like cellulose acetate, cellulose acetate trimellitate, cellulose acetate butyrate, cellulose acetate propionate, ethylcellulose, cetostearyl alcohol, cetyl alcohol, compritol® 888 ATO, precirol® ATO 5, gelucire® 50/13, eudragit® RSPO, eudragit® RLPO, stearic acid, carnauba wax and microcrystalline wax.

35. The glidants and lubricants of Further Embodiments 11-13, added are colloidal silicon dioxide, talc, magnesium stearate, stearic acid and sodium stearyl fumarate wherein glidant and lubricant present in dosage form is ranging from 0.5% to 3% w/w.

36. The solid dosage form of Further Embodiment 2, the uncoated core or capsule shell consists of active agent in a pure form, salt form or in complex form ranging from 25 mg to 300 mg and the active percentage weight may vary from 10% to 80% w/w of the dosage form, more specifically in the range of 15% to 60%.

37. The solid dosage form of Further Embodiment 2, wherein the core portion is suitably covered with either by a film layer and/or by an enteric layer with stabilizers added optionally to its coating solution to improve the stability of the dosage form by acting as a barrier to moisture or to control the drug release in a desired manner.

38. The core dosage form of Further Embodiment 11, is suitably film/sub coated with materials like hydroxypropyl cellulose, methacrylate copolymers like eudragit® E100 or eudragit® EPO, kollicoat® IR, opadry® II or opadry® AMB either directly or after mixing with suitable plasticizers, glidants and opacifying agents or coloring agents, wherein the coating solutions is prepared in aqueous, hydro-alcoholic or non aqueous solvent mixtures with stabilizer(s) optionally added to such coating solution.

39. The core dosage form of Further Embodiment 11, the enteric coat is applied either directly onto the core or applied over a film coat/subcoat consisting of materials like hypromellose phthalate, hypromellose acetate succinate, polyvinyl acetate phthalate based dispersions like Opadry® enteric and Sureteric® either directly or after mixing with suitable plasticizers, glidants, opacifying agents or coloring agents wherein the coating solutions is prepared in aqueous, hydro-alcoholic or non aqueous solvent mixtures with stabilizer(s) optionally added to such coating solution.

40. The stabilizers added to the coating step of Further Embodiment 37 are present either alone or in combination with butylated hydroxy anisole, butylated hydroxy toluene, toluene sulfonic acid, disodium edetate, edetic acid, maleic acid, lactic acid, propyl gallate, sodium formaldehyde sulfoxylate, benzoic acid, and aspartic acid.

41. The controlled release solid dosage form mentioned in Further Embodiment 1 is having the following dissolution profile for drug from uncoated/film coated bupropion HCl SR dosage form when tested at 37° C. in a USP type II apparatus, at 50 rpm in 900 mL in distilled water, after 1 hr the release is not less than 25%, after 4 hr is not more than 85%, after 8 hr is more than 80%.

42. The controlled release solid dosage form mentioned in Further Embodiment 1 exhibits the following dissolution profile for drug from uncoated/film coated bupropion HCl SR and bupropion HCl XR dosage forms when tested at 37° C. in a USP type II apparatus, at 75 rpm in 900 mL in 0.1N HCl buffer, the release after 1 hr is not more than 50%, after 4 hours is not more than 85%, after 8 hours is not less than 80%.

43. The controlled release solid dosage form mentioned in Further Embodiment 1 exhibits the following dissolution profile for drug from enteric coated bupropion HCl XR dosage form when tested at 37° C. in a USP type II apparatus, at 75 rpm in 0.1N HCl and phosphate buffer mixture conducted as per USP 27 for enteric coated dosage forms, wherein the release is less than 10% in 2 hr, not more than 60% in 4 hr, not less than 60% in 8 hr and not less than 80% in 16 hr.

44. The controlled release solid dosage form in Further Embodiment 1, wherein drug is stabilized by forming a complex with cation exchange resin present in the uncoated/film coated bupropion XR resinate dosage form exhibits the following dissolution profile for drug when tested at 37° C. in a USP type II apparatus, at 75 rpm in 900 mL of 0.1N HCl buffer or 0.2M phosphate buffer (pH 6.8), after 1 hr the release is not more than 50%, after 4 hours is not more than 85%, after 8 hr is not less than 80%.

45. The controlled release solid dosage form in Further Embodiment 1, wherein drug is stabilized by forming a complex with cation exchange resin present in the uncoated/film coated bupropion XR resinate dosage form that exhibits the following dissolution profile for drug when tested at 37° C. in a USP type II apparatus, at 75 rpm in 0.1N HCl and phosphate buffer mixture conducted as per USP 27 for enteric coated dosage form, wherein the release is less than 50% in 1 hr, not more than 85% in 4 hr and not less than 80% in 8 hr.

46. The controlled release solid dosage form in Further Embodiment 1, wherein drug is stabilized by forming a complex with cation exchange resin present in the enteric coated bupropion XR resinate dosage form that exhibits the following dissolution profile for drug when tested at 37° C. in a USP type II apparatus, at 75 rpm in 0.1N HCl and phosphate buffer mixture conducted as per USP27 for enteric coated dosage forms, wherein the release is less than 10% in 2 hrs, not more than 60% in 4 hr, not less than 60% in 8 hr and not less than 80% in 16 hr.

47. The solid dosage form mentioned in Further Embodiment 1, wherein the film coated/enteric coated dosage form stored at 40° C./75% RH for three months period maintains a minimum of 80% of the active drug.

48. The solid dosage form mentioned in Further Embodiment 1, wherein the active drug belongs to any of the following mentioned to provide sustained or controlled delivery or taste masking or stabilization of the drug as needed, the examples include any hydrochloride salt form of the drugs like amosulalol, betazolol, benazepril, bisoprolol, buspirone, barnidipine, bepridil, clofedanol, cetrizine, cephalexin, cyclobezaprine, dexamethylphenidate, esmolol, fluoxetine, flurazepam, fexofenadine, granisetron, hydralazine, loperamide, levamisole, lomefloxacin, metformin, methylphenidate, metoclopramide, mefloquine, nicardipine, ondansetron, oxycodone, oxymorphone, paroxetine, propafenone, quinapril, ritoridine, ranitidine, rimantadine, sertraline, ticlopidine, tolazoline, tamsulosin, tramadol, terbinafine, verapamil, valacyclovir, venlafaxine and other salt forms of drugs like abacavir sulfate, alendronate sodium, citalopram hydrobromide, clopidogrel bisulfite, cerivastatin sodium, enoxaprin sodium, fluvastatin sodium, fosinopril sodium, indinavir sulfate, losartan potassium, naproxen sodium, pravastatin sodium, piperacillin sodium, quetiapine fumarate, risedronate sodium, rosiglitazone maleate, valporate sodium, salbutamol sulfate, sumatriptan succinate, warfarin sodium and zolpidem tartrate.

It is to be understood that the invention is not limited to the exact details of operation, or to the exact compositions, methods, procedures or embodiments shown and described, as obvious modifications and equivalents will be apparent to one skilled in art, and the invention is therefore to be limited only by the full scope which can fairly, legally and equitably be accorded to the appended claims.

1. A pharmaceutical solid dosage form comprising buproprion hydrochloride and at least one member of the group consisting of: butylated hydroxyanisole, butylated hydroxytoluene and an ion exchange resin.

2. A pharmaceutical solid dosage form according to claim 1 and additionally comprising a glidant, a lubricant, a release-controlling agent, and optionally a pore-forming agent.

3. A pharmaceutical solid dosage form according to claim 1 and consisting essentially of buproprion hydrochloride, a glidant, a lubricant, a release-controlling agent, a diluent, and at least one member of the group consisting of butylated hydroxyanisole, butylated hydroxytoluene and an ion exchange resin.

4. A pharmaceutical solid dosage form according to claim 4 and wherein the glidant is Cab-o-sil; the lubricant is magnesium stearate; and the release-controlling agent is xanthan gum, hydroxyethyl cellulose, carnauba wax, Eudragit RSPO, Compritol ATO 888, Gelucire 50/13, cellulose acetate butyrate, polyvinyl alcohol, Kollidon VA64, ethyl cellulose, Kollidon SR, Polyox WSR 303, or hydroxypropyl cellulose.

5. A pharmaceutical formulation according to claim 1 comprising only one member of the group and wherein the member of the group is an ion exchange resin.

6. A pharmaceutical solid dosage form according to claim 5 and wherein the ion exchange resin is Amberlite IRP69.

7. A pharmaceutical solid dosage form according to claim 5 and further comprising at least one member of the group consisting of: a film coating and an enteric coating.

8. A pharmaceutical solid dosage form comprising a buproprion-resinate, a glidant, a lubricant, a release-controlling agent, and a diluent.

9. A pharmaceutical solid dosage form according to claim 8 and wherein the bupropion-resinate complex comprises Amberlite IRP69, the glidant is Cab-o-sil, the lubricant is magnesium stearate, and the release-controlling agent is hydroxypropyl cellulose, Eudragit RSPO, Polyox WSR 303, sodium carboxymethyl cellulose, hydroxyethyl cellulose, xanthan gum, Kollidon VA64, ethyl cellulose, or Kollidon SR.

10. A pharmaceutical solid dosage form according to claim 8 and further comprising at least one member of the group consisting of: a film coating and an enteric coating.

11. A pharmaceutical solid dosage form according to claim 1 and wherein the release of bupropion hydrochloride from the formulation occurs according to the limitation: when tested at 37° C. in a USP type II apparatus, at 50 rpm in 900 mL in distilled water, after 1 hr the release of bupropion hydrochloride from the formulation is not less than 25%, after 4 hr is not more than 85%, and after 8 hr is more than 80%.

12. A pharmaceutical solid dosage form according to claim 4 and wherein the release of bupropion hydrochloride from the formulation occurs according to the limitation: when tested at 37° C. in a USP type II apparatus, at 50 rpm in 900 mL in distilled water, after 1 hr the release of bupropion hydrochloride from the formulation is not less than 25%, after 4 hr is not more than 85%, and after 8 hr is more than 80%.

13. A pharmaceutical solid dosage form according to claim 7 and wherein the release of bupropion hydrochloride from the formulation occurs according to the limitation: when tested at 37° C. in a USP type II apparatus, at 75 rpm in 900mL in 0.1N HCl buffer, the release after 1 hr is not more than 50%, after 4 hours is not more than 85%, after 8 hours is not less than 80%.

14. A pharmaceutical solid dosage form according to claim 7 and wherein the release of bupropion hydrochloride from the formulation occurs according to the the limitation: when tested at 37° C. in a USP type II apparatus, at 75 rpm in 0.1N HCl and phosphate buffer mixture conducted as per USP 27 for enteric coated dosage forms, wherein the release is less than 10% in 2 hr, not more than 60% in 4 hr, not less than 60% in 8 hr and not less than 80% in 16 hr.

15. A pharmaceutical solid dosage form according to claim 8 and wherein the release of bupropion from the formulation occurs according to the limitation: when tested at 37° C. in a USP type II apparatus, at 75 rpm in 900 mL of 0.1N HCl buffer or 0.2M phosphate buffer (pH 6.8), after 1 hr the release is not more than 50%, after 4 hours is not more than 85%, after 8 hr is not less than 80%.

16. A pharmaceutical solid dosage form according to claim 10 and wherein the release of bupropion from the formulation occurs according to the limitation: when tested at 37° C. in a USP type II apparatus, at 75 rpm in 900 mL of 0.1N HCl buffer or 0.2M phosphate buffer (pH 6.8), after 1 hr the release is not more than 50%, after 4 hours is not more than 85%, after 8 hr is not less than 80%.

17. A pharmaceutical solid dosage form according to claim 8 and wherein the release of bupropion from the formulation occurs according to the limitation: when tested at 37° C. in a USP type II apparatus, at 75 rpm in 0.1N HCl and phosphate buffer mixture conducted as per USP 27 for enteric coated dosage form, wherein the release is less than 50% in 1 hr, not more than 85% in 4 hr and not less than 80% in 8 hr. Challapalli, Gumudavelli & Murty, Buproprion Formulation

18. A pharmaceutical solid dosage form according to claim 10 and wherein the release of bupropion from the formulation occurs according to the limitation: when tested at 37° C. in a USP type II apparatus, at 75 rpm in 0.1N HCl and phosphate buffer mixture conducted as per USP 27 for enteric coated dosage form, wherein the release is less than 50% in 1 hr, not more than 85% in 4 hr and not less than 80% in 8 hr.

19. A pharmaceutical solid dosage form according to claim 10 and wherein the release of bupropion from the formulation occurs according to the limitation: when tested at 37° C. in a USP type II apparatus, at 75 rpm in 0.1N HCl and phosphate buffer mixture conducted as per USP27 for enteric coated dosage forms, wherein the release is less than 10% in 2 hrs, not more than 60% in 4 hr, not less than 60% in 8 hr and not less than 80%in 16 hr.

20. A stabilized pharmaceutical composition comprising: (a) at least one member of the group of active ingredients consisting of buproprion and bupropion hydrochloride; and (b) at least one member of the group of stabilizers consisting of butylated hydroxyanisole, butylated hydroxytoluene, and an ion-exchange resin; and wherein (c) a tablet or a capsule containing from about 25 mg to about 300 mg of the active ingredient possesses at least one of the following profiles for the release of active ingredient: (i) when tested at 37° C. in a USP type II apparatus, at 50 rpm in 900 mL in distilled water, after 1 hr the release is not less than 25%, after 4 hr is not more than 85%, after 8 hr is more than 80%; (ii) when tested at 37° C. in a USP type II apparatus, at 75 rpm in 900 mL in 0.1N HCl buffer, the release after 1 hr is not more than 50%, after 4 hours is not more than 85%, after 8 hours is not less than 80%; (iii) when tested at 37° C. in a USP type II apparatus, at 75 rpm in 0.1N HCl and phosphate buffer mixture conducted as per USP 27 for enteric coated dosage forms, wherein the release is less than 10% in 2 hr, not more than 60% in 4 hr, not less than 60% in 8 hr and not less than 80% in 16 hr; (iv) when tested at 37° C. in a USP type II apparatus, at 75 rpm in 900 mL of 0.1N HCl buffer or 0.2M phosphate buffer (pH 6.8), after 1 hr the release is not more than 50%, after 4 hours is not more than 85%, after 8 hr is not less than 80%; (v) when tested at 37° C. in a USP type II apparatus, at 75 rpm in 0.1N HCl and phosphate buffer mixture conducted as per USP 27 for enteric coated dosage form, wherein the release is less than 50% in 1 hr, not more than 85% in 4 hr and not less than 80% in 8 hr.
